(19) United States
(12) Patent Application Publication
(10) Pub. No.: US 2025/0057448 A1
(43) Pub. Date: Feb. 20, 2025

(54) METHOD, DEVICE AND SYSTEM FOR DETERMINING A STATE OF HEALTH OF A PATIENT

(71) Applicants: HOCHSCHULE FULDA, Fulda (DE); UNIVERSITÄT AUGSBURG, Augsburg (DE)

(72) Inventors: Claudia Eberle, Leutkirch (DE); Christoph Ament, München (DE)

(21) Appl. No.: 18/722,950

(22) PCT Filed: Sep. 28, 2022
(86) PCT No.: PCT/EP2022/076936

§ 371 Date: Jun. 21, 2024

(30) Foreign Application Priority Data
Dec. 22, 2021 (DE) 10 2021 134 292.9

(51) Int. Cl. A61B 5/145; A61B 5/00; G16H 10/60; G16H 50/30

(52) U.S. Cl. CPC A61B 5/14532; A61B 5/4839; A61B 5/7246; G16H 10/60; G16H 50/30

(57) ABSTRACT

The present invention relates to a device (12) for determining a state of health of a patient (11), comprising: an input interface (16) for receiving a glucose measurement value with information relating to a glucose level of the patient and a glucose uptake input value with information relating to a glucose uptake of the patient; an estimation unit (18) for determining the state of health of the patient on the basis of the received values and a predefined state model, which represents the state of health of the patient by means of a state vector and using a plurality of model parameters, wherein the state vector comprises a glucose model value with information relating to the glucose level of the patient and can comprise an insulin model value with information relating to the insulin level of the patient; a comparison unit (20) for determining a deviation between the glucose measurement value and the glucose model value; and an individualization unit (22) for the patient-specific update of a model parameter on the basis of the determined deviation. Further model parameters can comprise individual glucose or insulin sensitivities, the incretin sensitivity and sensitivities with respect to lifestyle input values. The present invention also relates to a system (10) and to a method for determining a state of health of a patient (11).

The present invention relates to a device for determining a patient's state of health. The present invention further relates to a system and a method for determining a health status of a patient. In addition, the present invention relates to a computer program product.

Diabetes mellitus (DM), or in short diabetes (D), refers to a group of heterogeneous diseases, in particular metabolic disorders of carbohydrates, which include impaired glucose and insulin homeostasis and are associated with the common feature of chronic hyperglycemia (“hyperglycemia”). The metabolic disorders are based, for example, on a disturbance/dysfunction/resistance or deficiency of a patient's glucose and insulin homeostasis (corresponding to the underlying etiological form of diabetes) and can lead to chronic hyperglycemia in the absence of or inadequate treatment. Furthermore, other metabolic disorders, such as fat and protein metabolism and electrolyte disorders, are typical concomitant phenomena of diabetes resulting from the altered glucose and insulin homeostasis. The possible consequences of D, particularly if inadequately treated, include changes to the nervous and vascular systems, including the diabetic foot syndrome and coronary heart disease (CHD).

It should also be noted that impaired glucose tolerance (IGT), often referred to as a “precursor to diabetes mellitus”, can cause the above-mentioned concomitant and secondary diseases. IGT is characterized by an inadequate regulation of blood glucose, for example after carbohydrate intake, which is the basis of an early disturbance of glucose and insulin homeostasis. Blood glucose values measured at random are often still within the reference range, but in the course of a carbohydrate load (e.g. carbohydrate-rich meals, oral glucose tolerance test (oGTT), etc.) increased blood glucose values are found. The number of unreported cases of glucose tolerance disorders and D is very high, even worldwide.

The prevalence of D is steadily increasing worldwide. According to the International Diabetes Federation (IDF), around 539 million people worldwide have been diagnosed with D. Individual diabetes management, taking into account patient's individual circumstances, therefore plays a key role for patients themselves, healthcare providers and society as a whole.

There has been a fundamental change in measurement technology in recent years with the introduction of so-called (real-time) continuous glucose monitoring systems ((rt-)CGM) and/or (real-time) flash glucose monitoring systems ((rt-)FGM) and also combination approaches. In the past, patients diagnosed with diabetes were often only able to perform a few blood glucose measurements per day, whereby self-monitoring of blood glucose (SMBG) means a measurement by taking a blood sample. Monitoring metabolic dynamics or even diagnostic support using an algorithm was not possible or did not make sense on this basis. Today, (rt-)CGM and/or (rt-)FGM sensors have become inexpensive and suitable for everyday use, so that they are used by many patients diagnosed with diabetes. This makes dynamic glucose measurement data available with a considerably finer temporal resolution.

Also relevant in this context is the continuing spread of smartphones and other mobile devices, which are often used as personal hardware platforms. Nowadays, patients diagnosed with diabetes often use mobile devices to keep diabetic diaries and manage their data. However, automated evaluation and processing of the recorded data is still relatively uncommon. In most cases, the recorded data is merely displayed to the patient, but interpretation and self-management are still left to the patient or the treating doctor. The interaction between mobile devices and innovative measurement systems in particular offers the potential to provide patients with much more individualized and comprehensive support in real time.

In this context, a model-based diagnostic system is described in Eberle et al. “Real-time state estimation and long-term model adaptation: a two-sided approach toward personalized diagnosis of glucose and insulin levels”, 2012. In particular, real-time state estimation and long-term model parameter identification are pursued. It is described that real-time estimation of states and parameters enables improved state prediction and personalization of a model.

A disadvantage of previous approaches in this context is often the lack of individualization to the specific patient. Since patients differ greatly in terms of their metabolism, but also in terms of their reaction to external stimuli and their lifestyles, it is necessary to individualize a patient's state model. The more precisely these particular characteristics can be mapped, the better the patient can be supported in his or her decisions, for example with regard to insulin administration or food intake, in order to have as few restrictions as possible despite being diagnosed with D.

Based on this, the present invention sets itself the task of enabling precise modeling of a patient's state, in particular a state of health. In particular, a state of health should also be made precisely and accurately detectable for different patients.

To solve this problem, the present invention relates in a first aspect to a device for determining a state of health of a patient, comprising:
an input interface for receiving a glucose measurement value with information on a glucose level of the patient and a glucose uptake input value with information on a glucose uptake of the patient;
an estimation unit for determining the health state of the patient based on the received values and a predefined state model representing the health state of the patient by means of a state vector and using a set of model parameters, wherein the state vector comprises a glucose model value with information on the glucose level of the patient;
a comparison unit for determining a deviation between the glucose measured value and the glucose model value; and
an individualization unit for patient-specific updating of at least one model parameter based on the determined deviation.

In a further aspect, the present invention relates to a system for determining a health state of a patient, comprising a device as defined above and comprising a (real-time) continuous glucose monitoring system, (rt-)CGM, and/or a (real-time) flash glucose monitoring system, (rt-)FGM, for detecting a glucose measurement with information on a glucose level of the patient.

Other aspects of the invention relate to a method formed in accordance with the apparatus and a computer program product comprising program code for performing the steps of the method when the program code is executed on a computer. In addition, an aspect of the invention relates to a storage medium on which a computer program is stored which, when executed on a computer, causes execution of the method described herein.

Preferred embodiments of the invention are described in the dependent claims. It is understood that the features mentioned above and to be explained below can be used not only in the combination indicated in each case, but also in other combinations or on their own, without departing from the scope of the present invention. In particular, the method, the system and the computer program product may be implemented in accordance with the embodiments defined for the device in the dependent claims.

According to the invention, it is provided that a glucose measurement value and a glucose uptake value are received. The measured glucose value reflects a current glucose level of the patient. In particular, a measured value recorded shortly beforehand or provided in real-time by means of a corresponding measuring system can be received. The glucose uptake input value describes in particular an oral or intravenous glucose uptake by the patient. The glucose input value can, for example, be recorded by a sensor or provided by the patient via an interface.

Based on the two values received, a health state of the patient is determined in the estimation unit. This state of health is described by means of a state vector, whereby this state vector contains several state variables or states which, when viewed together or individually, enable a statement to be made regarding the patient's current state of health. The state model used is preferably discrete-time and the state vector is transferred from one point in time to a subsequent point in time using a predefined function, taking system inputs into account if necessary. The function is based on predetermined and modifiable model parameters or comprises such model parameters. In particular, the state vector provided according to the invention comprises a glucose model value which indicates the glucose level of the patient.

According to the invention, a deviation between the glucose measurement value received and the glucose model value estimated as part of the state vector is determined in the comparison unit. Based on the deviation between the two values, one of the model parameters is updated in the individualization unit on a patient-specific basis. In other words, it is proposed that at least one model parameter (which is not part of the state vector) is determined and updated online on the basis of a glucose measurement. In a model of a patient's glucose-insulin homeostasis, a model parameter is estimated.

As a result, an individualization of a model of the patient's state of health can be achieved, which creates an improved prediction and thus an improved possibility for the patient to exert influence. The patient-specific adaptation or updating of the model parameter proposed according to the invention can provide the patient with an accurate model of their state of health (also referred to as a digital twin). Based on this model, individualized therapy suggestions or individualized recommendations for action can then be determined in automated form and made available to the patient or the practitioner. Overall, this results in improved treatment options, increased quality of life for patients and a reduction in diabetes-associated co-morbidities and mortality.

In a preferred embodiment, the state vector comprises an insulin model value with information on an insulin level of the patient. The multiple model parameters comprise sensitivity parameters that represents a glucose and/or insulin sensitivity of the patient. The individualization unit is configured to update the sensitivity parameters. In particular, the approach according to the invention can be used in the DM environment. The state vector comprises an insulin model value and the model is based on a model parameter that represents glucose and/or insulin sensitivities of the patient. These sensitivities often vary from patient to patient. By individualizing this model parameter, the state model can be adapted to a patient. The prognostic capacities by means of the state estimation proposed according to the invention is improved.

In a preferred embodiment, the multiple model parameters comprise an incretin effect sensitivity parameter that maps a sensitivity of the patient's insulin level to a glucose uptake. The individualization unit is configured to update the incretin effect sensitivity parameter. A further individualization can be an adjustment of a patient-specific incretin sensitivity of the patient. In particular, a sensitivity of the insulin level to glucose uptake is mapped. Such a sensitivity can differ, for example, due to the patient's body weight or their metabolic characteristics.

In a preferred embodiment, the individualization unit is configured to update the model parameters based on a predefined cost function. It is also possible to use a cost function in order to be able to make a selection when updating the model parameters. The cost function can be used, for example, to prioritize different individualization options.

In a preferred embodiment, the individualization unit is configured to determine a gradient with regard to the model parameters and to update the model parameters using a gradient method. In particular, it is possible that a gradient method is used in which, for example, an update is carried out in the direction of a steepest descent (or ascent, depending on the sign definition). A gradient method is a method for solving optimization problems. In particular, an optimization is to be carried out in such a way that the best possible representation of the real glucose measurement values and the estimated glucose model value is achieved by adjusting the model parameters. This results in an individual adaptation of the modeling to a patient.

In a preferred embodiment, the estimation unit is configured to update the state vector based on a previous state vector and based on a further predefined cost function and a further gradient method. It is possible that a gradient method is also used when updating the state vector. In this respect, a predefined cost function is also used when updating the state vector.

In a preferred embodiment, the predefined state model is a nonlinear differential equation model. In particular, the predefined state model can be a nonlinear differential equation model of order 9 or higher. The use of a nonlinear differential equation model makes it possible to map the state transitions and the states with a high degree of accuracy, so that accurate prediction is possible. This results in a high degree of precision in mapping the patient's state of health.

In a preferred embodiment, the input interface is configured to receive the glucose measurement value from a (real-time) continuous glucose monitoring system, (rt-)CGM, and/or a (real-time) flash glucose monitoring system, (rt-) FGM. In particular, the input interface can be configured to receive the glucose measurement via a wireless communication link. The (rt-)CGM and/or (rt-)FGM systems widely used today often provide for data transmission via wireless connection. For example, the device according to the invention can then be implemented in the form of a smartphone or in the form of a smartphone app in order to be able to process the corresponding sensor data. Communication via the internet (cloud-based) is also conceivable. This results in a convenient application of the device according to the invention.

In a preferred embodiment, the input interface is configured to receive an additional input value comprising information on the patient's diet, exercise status, sleep, medication and/or a pre-existing or concomitant disease. In other words, so-called lifestyle parameters can be received as additional information, which can then be taken into account when determining the patient's state of health. Such additional input values often enable a further improvement in the accuracy of the prediction of the person's state of health.

In a preferred embodiment, the multiple model parameters comprise at least one additional sensitivity parameter that represents a sensitivity of the patient's diet, exercise status, sleep, medication and/or a pre-existing or concomitant disease. The individualization unit is configured to update at least one additional sensitivity parameter. In other words, the various individual lifestyle parameters are taken into account in order to be able to map the patient's sensitivity to the various parameters. Different patients react differently to, for example, lack of sleep or changes in diet. These phenomena can be mapped by taking into account the additional input values or by using at least one additional sensitivity parameter. This results in further improved accuracy.

In a preferred embodiment, the device according to the invention comprises a dosing unit for determining a drug dosage in the context of a diabetes therapy based on the state vector. The dosing unit is preferably configured to determine the medication dosage based on a model predictive control (MPC) approach, a proportional-integral-differential controller (PID controller), a fuzzy controller and/or a deep learning approach. In particular, an automated determination of a medication dosage can be made based on the determined state. In particular, an insulin dosage can be recommended. PID controllers, fuzzy controllers, model predictive control (MPC) or deep learning approaches can be used as control algorithms, for example. For the patient, the effort involved in determining his or her insulin requirement is reduced. In addition, susceptibility to errors can be reduced. Health-threatening situations and co-morbidities as well as inpatient admissions can be avoided. Specifically, it is therefore possible, for example, that the dosing unit is configured to control an insulin pump, a smart pen or other devices for administering insulin.

In a preferred embodiment, the input interface is configured to receive an insulin input value with information on a current insulin dosage of the patient. The dosing unit is configured to determine a new insulin dosage based on the state vector and the insulin input value. By additionally taking the patient's insulin dosage into account, a suggestion for a new insulin dosage can be determined for diabetics. This results in a further reduction in the susceptibility to errors. In addition, health-threatening situations, co-morbidities and hospital admissions can be avoided.

In this context, a patient's state of health is understood in particular as a representation of a state by various variables that are summarized in a state vector. These variables enable a statement to be made about the patient's health, for example by comparing them with normal or average values or by evaluating deviations, etc. In particular, a state model is understood to be a discrete-time modeling of the state of health based on the state vector and a function by which this state vector can be transferred from one point in time to a subsequent point in time. According to the invention, several so-called model parameters are involved or included in this function. In particular, these model parameters can be values that map the sensitivities of a patient to certain inputs.

The state model maps a state to a state at a subsequent point in time by means of this function, taking into account received input values. A patient-specific update is understood here as a change to one of the model parameters by which this model parameter is adapted to a characteristic of an individual.

The invention is described and explained in more detail below with reference to some selected embodiments in connection with the accompanying drawings. It shows:

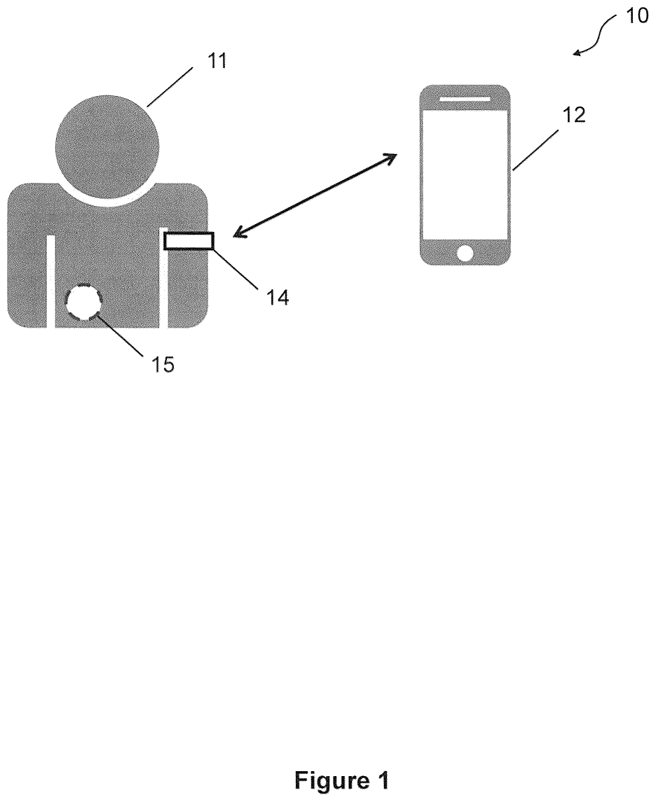

FIG. 1 a schematic representation of a system according to the invention for determining a patient's state of health;

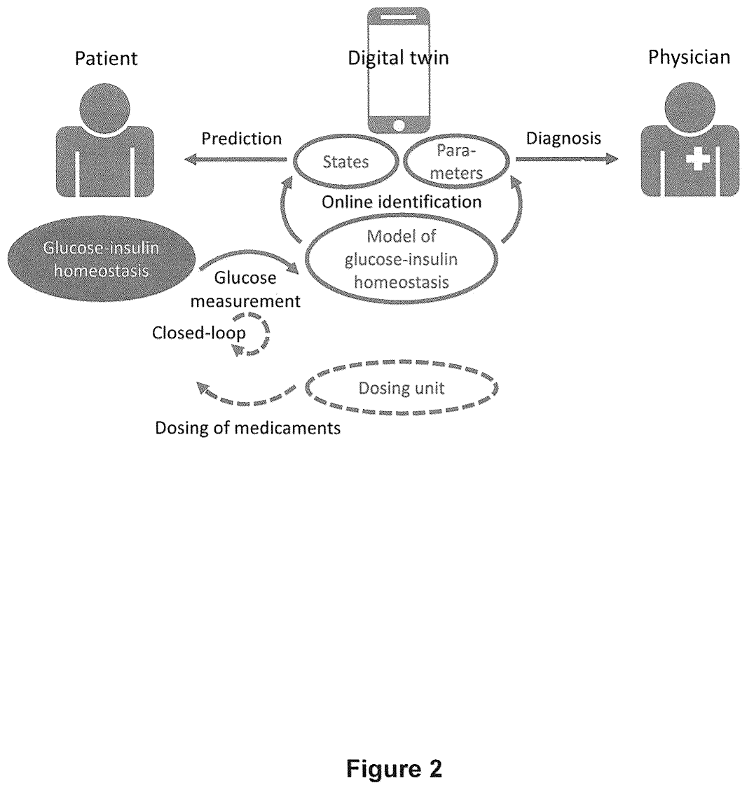

FIG. 2 a schematic overview of a determination of states and parameters of a glucose-insulin model according to one embodiment of the approach of the present invention;

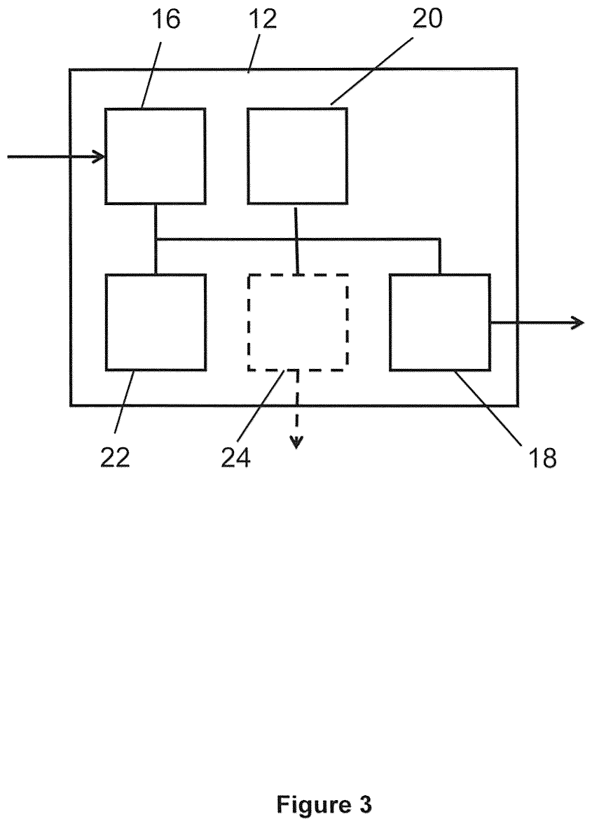

FIG. 3 a schematic representation of a device according to the invention;

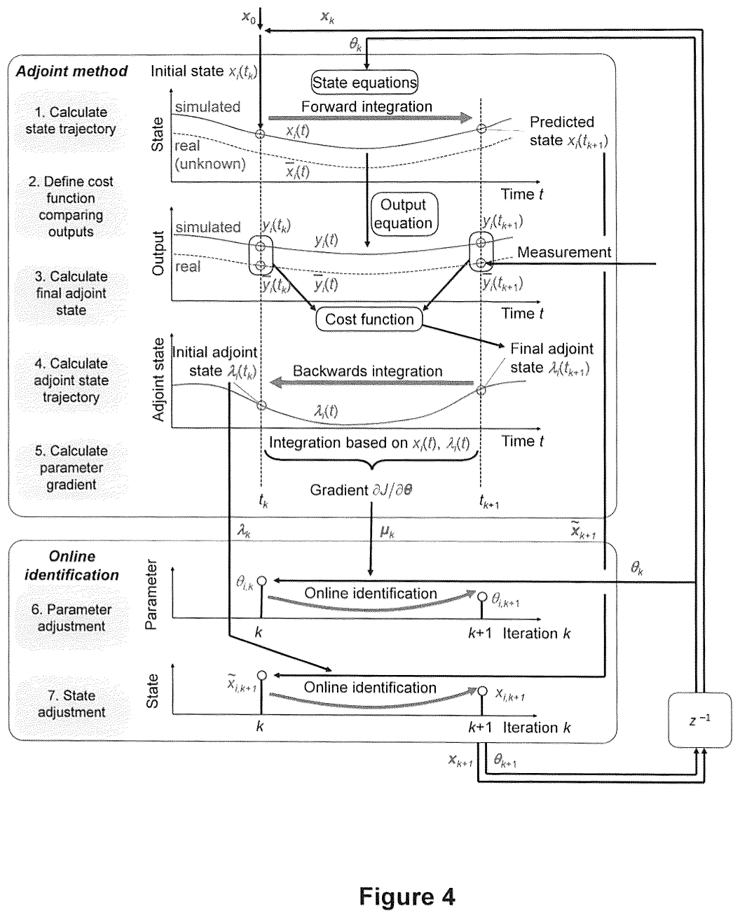

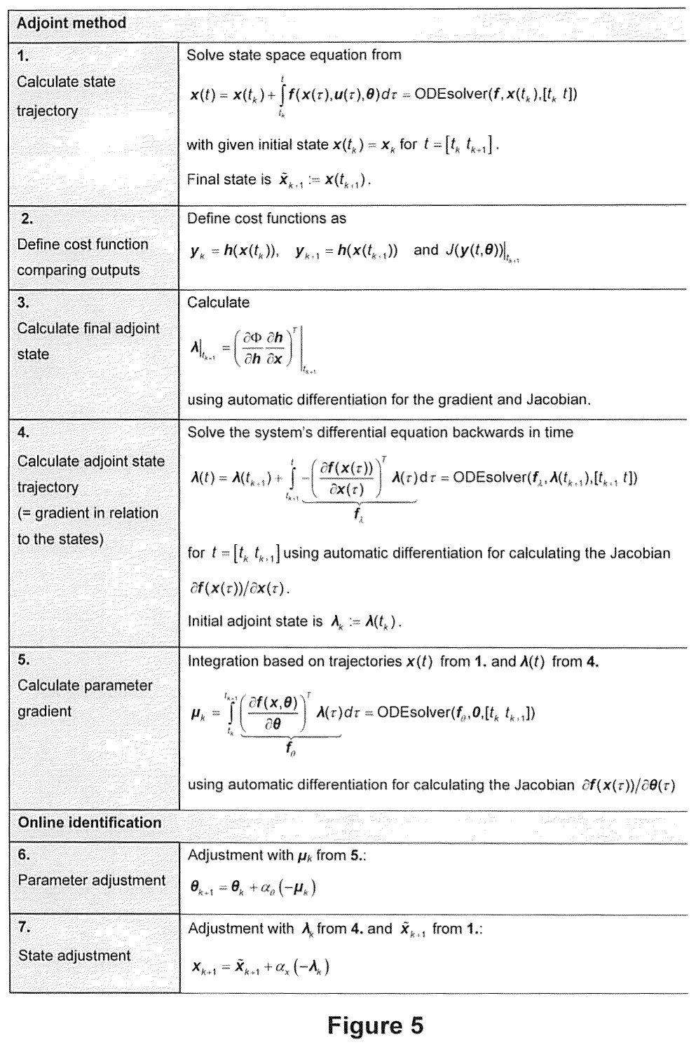

FIGS. 4 and 5 a schematic representation of an embodiment of the algorithm proposed according to the invention for parallel tracking of parameters and states; and

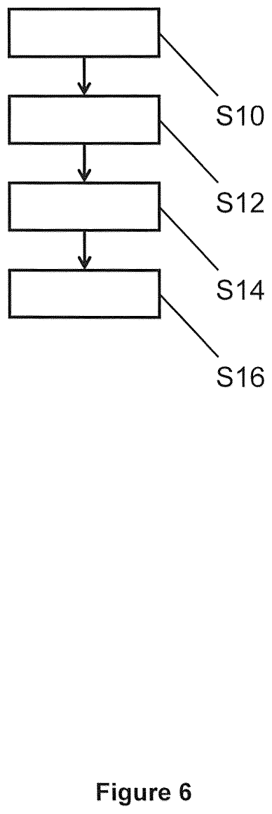

FIG. 6 a schematic representation of the method according to the invention for determining a patient's state of health.

FIG. 1 schematically illustrates a system 10 according to the invention for determining the state of health of a patient 11. The system 10 comprises a device 12 for determining the state of health and a continuously measuring glucose sensor 14 ((rt-)CGM and/or (rt-)FGM sensor). The illustration is to be understood as a schematic overview. In the illustrated embodiment example, the device 12 is configured as a smartphone that communicates with the continuously measuring glucose sensor 14, which is attached by means of a wristband or by means of an adhesive, via Bluetooth connection and receives a glucose measurement value from it. It is understood that other transmission paths are also possible. It is also understood that the system 10 can also comprise, for example, an (optional) insulin pump 15, which can be controlled directly, for example, also via a Bluetooth connection from the smartphone or via the Internet in a cloud-based or other approach.

The approach according to the invention is particularly aimed at individualized diabetes therapy. In previous approaches, measurements from (rt-)CGM and/or (rt-)FGM sensors have already been processed in models to characterize the glucose-insulin homeostasis of patients diagnosed with diabetes. According to the invention, it is proposed to create a precise dynamic model of the glucose-insulin homeostasis of an individual patient.

As shown in FIG. 2, model states and parameters are to be continuously adapted on the basis of online glucose measurements (glucose measurement value), which enable individual predictions and diagnoses to facilitate diabetes self-management of a patient diagnosed with diabetes.

As shown by the dotted lines, direct control for dosing medication, in particular an insulin pump, is optionally possible (closed-loop control).

In this respect, the system or device proposed according to the invention is aimed to extend automation in the sense of closed-loop control of an insulin pump “automated insulin delivery (AID) systems” or “artificial pancreas”), in which an insulin pump is controlled by means of the model directly based on measurements and other inputs.

FIG. 3 schematically illustrates a device 12 according to the invention for determining a patient's state of health. The device comprises an input interface 16, an estimation unit 18, a comparison unit 20 and an individualization unit 22. In the illustrated embodiment example, the device 12 also comprises an (optional) dosing unit 24. The units and interfaces can be implemented partially or completely in software and/or hardware. In particular, the units can be configured as a processor, processor modules or also as software for a processor. The device 12 can be configured in particular in the form of a smartphone or another mobile device or as software or an app for a smartphone or a mobile device.

It is possible for parts of the device to be made available as functionalities of a central server via the Internet (cloud-based). For example, a patient can use a smartphone app to communicate with a server that is configured to provide all or part of the functions of the above-mentioned units. This is particularly advantageous if other components such as a CGM/FGM or an insulin pump or other components that are also internet-enabled are included. Communication between different components of a system according to the invention can then take place via the Internet.

A glucose measurement value and a glucose uptake input value are received via the input interface 16. The measured glucose value can be received directly from a corresponding (rt-)CGM and/or (rt-)FGM sensor via a Bluetooth connection, for example. In particular, the glucose reading indicates a current glucose level of the patient. The glucose uptake input value can, for example, be based on an input from the patient or also on a measurement by a corresponding sensor and indicates a glucose uptake of the patient. For example, the glucose uptake input value can be used to indicate the amount of glucose the patient has consumed in a predefined period of time in the past.

In further embodiments of the device 12 according to the invention, it is also possible for additional parameters to be received. In particular, so-called lifestyle parameters can be received, for example, parameters that comprise as additional input values, for example, information on a diet, an exercise status, sleep, medication and/or a pre-existing or concomitant disease of the patient. These additional input values can also be taken into account in the further modeling of the patient's state of health. The format of a corresponding additional input value can be selected individually. In particular, the values can be specified on a predefined scale. It is also conceivable that an assignment is made using a corresponding table so that, for example, a patient's exercise or exercise status is classified into categories (e.g. no sport, sport once or twice a week, sport three to four times a week, sport more than five times a week, etc.).

In the estimation unit 18, a health status of the patient is determined based on the values received. A predefined state model is used for this purpose, in which the patient's state of health is mapped by means of a state vector. The estimation unit can transmit the state of health or the state vector to a corresponding display device, for example. It is also conceivable that the estimation unit 18 is in contact via a mobile communication link with a medical professional, for example, who evaluates the patient's current estimated state of health and can provide therapy recommendations based on this, for example. In this respect, the estimation unit 18 forms a kind of digital replica (digital twin) of the patient. The patient, but also other interested parties, can gain an insight into the patient's current state of health. In particular, it is possible to directly determine an individual medication dosage for diabetes therapy based on the state vector.

The estimation unit 18 can, for example, use a predefined cost function and a gradient method to update the state vector. This optimizes a mathematical model, which in turn is used to update the state vector from one time step to the next. Different models can be used for this purpose.

The predefined state model in the estimation unit 18 can in particular be a nonlinear differential equation model, especially of order 9 or higher. This enables a reliable and accurate estimation of the state transition.

In particular, a state vector is used in the estimation unit 18, which also comprises a glucose model value. This glucose model value simulates the patient's glucose level and, in this respect, comprises information on the patient's glucose level. A predefined scale can be used for this purpose. For example, it is conceivable to specify a percentage value.

A deviation between the measured glucose value and the glucose model value is now determined in the comparison unit 20. The deviation can be determined in particular in the form of an absolute difference.

Based on this deviation, at least one model parameter is updated in the individualization unit 20. It is possible for a model parameter to represent a sensitivity parameters that maps a glucose and/or insulin sensitivities of the patient. In particular, it is further possible that the state vector comprises an insulin model value indicating an insulin level of the patient. The model parameters may comprise sensitivity parameters that maps a glucose and/or insulin sensitivity of the patient. It may then be provided that the individualization unit 22 is configured to adapt these sensitivity parameters and thereby achieve a patient-specific adaptation of the model. Each patient has individual sensitivities with regard to the administration of glucose or insulin. These individual sensitivities can be mapped to this extent, resulting in improved modeling. It is also possible to use an incretin effect sensitivity parameter, which maps the sensitivity of the patient's insulin level to glucose uptake. It is then possible for the individualization unit 22 to update this incretin effect sensitivity parameter in order to achieve a patient-specific adaptation of the model. The individualization unit 22 can also use a predefined cost function. In particular, a gradient method may be used to update the model parameters.

In the optional dosing unit 24, a medication dosage can be determined directly based on the status vector. In particular, it is advantageous if medication is even dispensed directly based on this. For example, an insulin dosage can be determined and an insulin pump can be controlled directly. Automated insulin delivery (AID) has become increasingly relevant in recent years and offers relevant relief for patients.

The algorithm proposed herein for mapping the state of health of a patient by means of a state model preferably works in discrete time steps k. The time step width is determined in particular by the cycle of the glucose measurement. (rt-) CGM and/or (rt-)FGM sensors typically output a new measured value yₖ every 1-5 minutes, after which the algorithm is run once to update states xₖ and model parameters θₖ

States xₖ are the time-varying signals of the (state) model, such as the glucose or insulin concentration in the blood or tissue. The states are summarized in the state vector. There are two main mechanisms for updating: In the first step, all states are considered in a model of glucose-insulin homeostasis. It is therefore possible to update them by simulating the model. For example, if it is known that food intake is currently taking place (through the glucose uptake input value), the model will predict an increase in blood glucose. In the second step, the model output is compared with the current measurement. If a deviation occurs, the states are corrected. For this purpose, this deviation is evaluated in the algorithm, in particular with the help of a cost function Jₖ, for which the gradient λₖ is determined with regard to the states xₖ. This gradient can then be used in a gradient method to correct the state predicted on the basis of the model.

The time-invariant constants of the state model are referred as model parameters θₖ. In particular, the state model can be a model of glucose-insulin homeostasis. The model parameters are, for example, time constants that describe the dynamics of an insulin reaction or glucose degradation, or amplifications such as insulin or glucose sensitivities. These model parameters are obviously highly dependent on the individual patient. Therefore, updating the model parameters allows individualization of the model. In this way, it is possible to learn from past measurements. This mechanism is analogous to the second step of updating the states described above: In particular, the same cost function Jₖ is used to evaluate deviations between model output and measurement. However, the gradient μₖ is now determined with regard to the model parameters θₖ. This gradient is used with a separate gradient method to correct the model parameters. The algorithm is shown in detail in FIGS. 4 and 5.

The current state xₖ offer the patient real-time information on his or her metabolic (diabetic) state of health. This information is more comprehensive than just the blood glucose measurement.
This can be used for self-management of diabetes and alarms can be triggered if necessary.
On this basis, predictions can be made to support treatment planning (see FIG. 2). For example, the course of blood glucose in the next 120 minutes can be predicted based on the current condition.
In the future, a closed-loop control with an insulin pump could benefit from the comprehensive state information as part of automated insulin delivery AID (“artificial pancreas”).

The individualized model parameter ex allows a better tailored representation of the patient in the model of glucose-insulin homeostasis in the sense of a “digital twin”.
In particular, this improves the model-based updating of the state in the first step described above.
In addition, the model parameters obtained can be used for diagnosis (see FIG. 2). A decrease in insulin sensitivity, for example, can indicate a worsening of the diabetic condition.

The automatic differentiation method (using dual numbers) is used to calculate the two gradients (see, for example, R. D. Neidinger, Introduction to Automatic Differentiation and MATLAB Object-Oriented Programming, SIAM Rev. 52 (2010) 545-563; and D. Maclaurin, D. Duvenaud, M. Matt Johnson, J. Townsend, autograd: Software package that automatically differentiate native Python and Numpy code, 2020). This means that no analytical or symbolic derivatives need to be determined. The method offers an exact solution and is therefore also advantageous compared to approximation methods such as calculation via difference quotients.

In order to model the individual dynamic metabolic system, a nonlinear differential equation model of order n=9 was developed (corresponding to 9 modeled compartments), which is based on the approach of Bergman et al. (see R. N. Bergman, Y. Z. Ider, C. R. Bowden, C. Cobelli, Quantitative estimation of insulin sensitivity, American Journal Physiology-Endocrinology and Metabolism 236 (1979 E667). Cobelli, Quantitative estimation of insulin sensitivity, American Journal of Physiology-Endocrinology and Metabolism 236 (1979) E667), but takes other influences such as oral food intake into account (see table below, where the non-individual standard model parameters and input signals are given in the right-hand column of the differential equations). It is less complex than the model of Cobelli et al. (see C. Dalla Man, R. A. Rizza, C. Cobelli, Meal simulation model of the glucose-insulin system, IEEE Trans. Biomed. Eng. 54 (2007) 1740-1749; and C. Cobelli, C. D. Man, G. Sparacino, L. Magni, G. de Nicolao, B. P. Kovatchev, Diabetes: Models, Signals, and Control, IEEE Rev. Biomed. Eng. 2 (2009) 54-96) and is therefore suitable for online simulation on smaller (mobile) platforms.

| Compartment | Differential equation |
|---|---|
| 1. plasma glucose G(t) in mg/dl | $\frac{dG ( t )}{dt} = - \frac{1}{{T}_{G}} G ( t ) - {k}_{X} X ( t ) [ G ( t ) + {G}_{basal} ] + {q}_{G 2} ( t ) + {u}_{GV} ( T )$ ${T}_{G} = 32.45 min : Time constant of glucose degradation \\ {k}_{X} = 507 {10}^{- 6} ml / μU - mg / dl / min : Insulin sensitivity \\ {G}_{basal} = 90 mg / dl : Basal plasma glucose \\ {u}_{GV} Input signal in mg / dl / min : Venous glucose uptake input$ |
| 2. plasma insulin I(t) in μU/ml | $\frac{dI ( t )}{dt} = - \frac{1}{{T}_{I}} I ( t ) + {v}_{1} ( t ) + {v}_{2} ( t ) + {k}_{G 3} {q}_{G 1} ( t ) + {q}_{I} ( t )$ ${T}_{I} = 3.33 min : Time constant of insulin degradation \\ {k}_{G 3} = 2. μU / ml dl / mg : amplification of the incretin effect$ |
| 3. interstitial insulin X(t) in μU/ml | $\frac{dX ( t )}{dt} = - \frac{1}{{T}_{X}} X ( t ) + \frac{1}{{T}_{X}} I ( t )$ ${T}_{X} = 47.78 min : Intertitial insulin time constant$ |
| 4. interstitial glucose Y(t) in mg/dl | $\frac{dY ( t )}{dt} = - \frac{1}{{T}_{Y}} Y ( t ) + \frac{1}{{T}_{Y}} G ( t )$ ${T}_{Y} = 16. min : Interstitial glucose time constant$ |
| 5. phase 1 of the glucose reaction v₁ (t) in μU/ml/min | $\frac{{dv}_{1}^{′} ( t )}{dt} = - \frac{1}{{T}_{1}} {v}_{1}^{′} ( t ) - \frac{{k}_{G 1}}{{T}_{1}^{2}} G ( t ) , with {v}_{1} ( t ) = {v}_{1}^{′} ( t ) + \frac{{k}_{G 1}}{{T}_{1}} G ( t )$ ${T}_{1} = 4. min : Time constant of phase 1 \\ {k}_{G 1} = 1 , 080 dl / mg - μU / ml : Glucose sensitivity ( phase 1 )$ |
| 6. phase 2 of the glucose reaction v₂ (t) in μU/ml/min | $\frac{{dv}_{2} ( t )}{dt} = - \frac{1}{{T}_{2}} {v}_{2} ( t ) + \frac{{k}_{G 2}}{{T}_{2}} G ( t )$ ${T}_{2} = 12. min : Time constant of phase 2 \\ {k}_{G 2} = 0.107 dl / mg μU / ml / min : Glucose sensitivity ( phase 2 )$ |
| 7. oral glucose flow (1) q_{G1} (t) in mg/dl/min | $\frac{{dq}_{G 1} ( t )}{dt} = - \frac{1}{{T}_{O 1}} {q}_{G 1} ( t ) + \frac{1}{{T}_{O 1}} {u}_{GO} ( t )$ ${T}_{O 1} = 15. min : Oral time constant 1 \\ {u}_{GO} Input signal in mg / dl / min : Oral glucose uptake input$ |
| 8. oral glucose flow (2) q_{G2} (t) in mg/dl/min | $\frac{{dq}_{G 2} ( t )}{dt} = - \frac{1}{{T}_{O 2}} {q}_{G 2} ( t ) + \frac{1}{{T}_{O 2}} {q}_{G 1} ( t )$ ${T}_{O 2} = 60. min : Oral time constant 2$ |
| 9. subcutaneous insulin flow qₗ (t) in μU/ml/min | $\frac{{dq}_{I} ( t )}{dt} = - \frac{1}{{T}_{S}} {q}_{I} ( t ) + \frac{1}{{T}_{S}} {u}_{IS} ( t )$ ${T}_{S} = 10. min : time constant of the subcutaneous insulin \\ {u}_{IS} Input signal in μU / ml / min : Subcutaneous insulin dosage$ |

The state vector x and the input vector u are defined as

$$\begin{matrix}x ( t ) = {[ \begin{matrix}G ( t ) & I ( t ) & X ( t ) & Y ( t ) & {v}_{1} ( t ) & {v}_{2} ( t ) & {q}_{G 1} ( t ) & {q}_{G 2} ( t ) & {q}_{I} ( t )\end{matrix} ]}^{T} \\ u ( t ) = {[ \begin{matrix}{u}_{GV} ( t ) & {u}_{IS} ( t ) & {u}_{GO} ( t )\end{matrix} ]}^{T}\end{matrix}$$

Hence, the differential equations from the table above can be combined to form the state differential equation:

$$\frac{dx ( t )}{dt} = f ( x ( t ) , u ( t ) , θ )$$

The model parameters to be identified are separated and form the parameter vector θ.

Starting from state x, the output y is defined in general form by the output equation:

$$y ( t ) = h ( x ( t ) )$$

If the interstitial glucose is measured by an (rt-)CGM and/or (rt-)FGM sensor, the following applies:

$$y ( t ) = {x}_{4} ( t ) = Y ( t )$$

If the plasma glucose is recorded, the following applies:

$$y ( t ) = {x}_{1} ( t ) = G ( t )$$

Finally, a suitable cost function Jₖ must assess the deviation between the simulated output y and the measured output y̅. Preferably, both a local and a global error are defined and introduced into the cost function by a weighing matrix Q (as a diagonal matrix with positive weights):

$$\begin{matrix}J ( y ( t , θ ) ) \vert {t}_{k + 1} = \begin{matrix}\frac{1}{2} {e}^{T} ( θ ) & Q & e ( θ ) & mit & e ( θ ) = [ \begin{matrix}{e}_{lokal} ( θ ) \\ {e}_{global} ( θ )\end{matrix} ]\end{matrix} \\ = [ \begin{matrix}( {\overline{y}}_{k + 1} - {\overline{y}}_{k} ) - ( {y}_{k + 1} ( θ ) - {y}_{k} ( θ ) ) \\ {\overline{y}}_{k + 1} - {y}_{k + 1} ( θ )\end{matrix} ]\end{matrix}$$

According to the invention, a simultaneous estimation of states and model parameters is thus proposed. The adjoint method used here is mostly used for the design of optimal controls. However, it can also be used for parameter identification, which has recently been successfully demonstrated in relation to neural networks.

The method presented here is also based on this, but extends it with a second gradient method for the parallel estimation of the states. Our own investigations to date show that this also benefits the parameter estimation.

State estimation can be realized by Bayes estimators (e.g. Kalman filters), but this requires additional effort. Special solutions must be found for nonlinear systems in particular, whereas parallel estimation is also directly suitable for nonlinear systems and can be carried out from a single approach.

Model predictive control (MPC) is a well-known approach for regulating insulin dosage (“closed-loop” or “artificial pancreas”). Model-based diagnosis with a complete metabolic model has not yet been used.

So far, no individual models have been used. Due to the great variability between patients, this is a significant shortcoming. Approaches to take this into account can be found in the context of MPC, but these often only switch between three basic models, so that only a relatively rough adjustment is achieved.

Based on tests with real measurement data, a selection of the model parameters to be identified in θ and the learning rates α_{q} and αₓ can be determined.

With regard to possible applications of the invention, for example, all applications may be considered that benefit from an improved state and parameter estimation within a model of glucose-insulin homeostasis in the context of a diabetes diagnosis or therapy. Examples:
Diabetes companion: Provides a more comprehensive diagnosis of the current status compared to just displaying the measured glucose value. Feedback of the current status to the patient, alarms if necessary.
Algorithm-based prediction and training: Predictions can be performed starting from the current state and different scenarios (e.g. for different meals) can be considered. Such simulations can also support patient training.
Closed-loop insulin therapy (“artificial pancreas”): A future automatic regulation of insulin could also work more precisely thanks to an improved diagnosis of the algorithm.
Tracking the state of health: The model parameters of the individual model can be used to assess the state of health. This is done retrospectively and trends can be derived.

The aforementioned applications can be implemented in products. These are stand-alone software products (diabetes companion as an app, training or health status tracking on a PC) or embedded solutions for existing hardware (smart CGM sensor or smart insulin pump).

FIG. 6 schematically illustrates a method according to the invention for determining a health state of a patient. The method comprises steps of receiving S10 a glucose measurement value and a glucose uptake input value, determining S12 the patient's state of health, determining S14 a deviation and updating S16 a model parameter on a patient-specific basis. The method can be implemented, for example, in the form of an app that can be executed on a mobile device, in particular a smartphone. As already described above, it is also possible for the steps to be performed in whole or in part by a corresponding server or another system in the sense of a cloud approach and made available as a service via the Internet.

The invention has been comprehensively described and explained with reference to the drawings and the description. The description and explanation are to be understood as examples and not limiting. The invention is not limited to the disclosed embodiments. Other embodiments or variations will be apparent to those skilled in the art from the use of the present invention and from a detailed analysis of the drawings, the disclosure and the following claims.

In the claims, the words “comprising” and “with” do not exclude the presence of further elements or steps. The undefined article “one” or “a” does not exclude the presence of a plurality. A single element or unit may perform the functions of more than one of the units recited in the claims. An element, a unit, an apparatus and a system may be partially or fully implemented in hardware and/or in software. The mere mention of some measures in several different dependent claims is not to be understood as meaning that a combination of these measures cannot also be used advantageously. A computer program can be stored/distributed on a non-volatile data carrier, for example on an optical memory or on a solid-state drive (SSD). A computer program can be distributed together with hardware and/or as part of hardware, for example by means of the Internet or by means of wired or wireless communication systems. Reference signs in the patent claims are not to be understood restrictively.

1. A device for determining a state of health of a patient, comprising:
an input interface for receiving a glucose measurement value with information on a glucose level of the patient and a glucose uptake input value with information on a glucose uptake of the patient;
an estimation unit for determining the state of health of the patient based on the received values and a predefined state model that maps the state of health of the patient by means of a state vector and using a set of model parameters, wherein the state vector comprises a glucose model value with information on the glucose level of the patient;
a comparison unit for determining a deviation between the glucose measurement value and the glucose model value;
and an individualization unit for updating at least one model parameter for each patient individually based on the determined deviation.

2. The device according to claim 1, wherein the state vector comprises an insulin model value with information on an insulin level of the patient and the set of model parameters comprises sensitivity parameters that reflect a glucose and/or insulin sensitivity of the patient; and the individualization unit is configured to update the sensitivity parameters.

3. The device according to claim 2, wherein the set of model parameters comprises an incretin effect sensitivity parameter that maps a sensitivity of the insulin level of the patient to a glucose uptake; and the individualization unit is configured to update the incretin effect sensitivity parameter.

4. The device according to claim 1, wherein the individualization unit is configured to update the model parameters based on a predefined cost function.

5. The device according to claim 4, wherein the individualization unit is configured to determine a gradient with respect to the model parameters and to update the model parameters using a gradient method.

6. The device according to claim 1, wherein the estimation unit is configured for updating the state vector based on a previous state vector and based on another predefined cost function and another gradient method.

7. The device according to claim 1, wherein the predefined state model is a nonlinear differential equation model, in particular a nonlinear differential equation model of order 9 or higher.

8. The device according to claim 1, wherein the input interface is configured to receive the glucose measurement value from a real-time continuously glucose monitoring system, (rt-)CGM, and/or a real-time flash glucose monitoring system, (rt-)FGM, in particular via a wireless communication connection.

9. The device according to claim 1, wherein the input interface is configured to receive an additional input value which comprises information on a diet, an exercise status, a sleep, a medication and/or a previous or concomitant illness of the patient.

10. The device according to claim 9, wherein the set of model parameters comprises at least one additional sensitivity parameter which represents a sensitivity of the patient to a diet, an exercise status, a sleep, a medication and/or a previous or concomitant disease; and the individualization unit is configured to update at least one additional sensitivity parameter.

11. The device according to claim 1 with a dosing unit for determining a medication dosage in the context of diabetes therapy based on the state vector, wherein the dosing unit is configured to determine the medication dosage based on a model predictive control (MPC) approach, a proportional-integral-derivative (PID) controller, a fuzzy controller and/or a deep learning approach.

12. The device according to claim 11, wherein the input interface is configured to receive an insulin input value with information on a current insulin dosage of the patient; and the dosing unit is configured to determine a new insulin dosage based on the state vector and the insulin input value.

13. A system for determining a health state of a patient, comprising:
a device according to claim 1;
a real-time continuously glucose monitoring system, (rt-)CGM, and/or a real-time flash glucose monitoring system, (rt-)FGM, for recording the glucose measurement value; and
a user interface for recording the glucose uptake input value.

14. A method for determining a health status of a patient, comprising:
receiving a glucose measurement value with information on a glucose level of the patient and a glucose uptake input value with information on a glucose uptake of the patient;
determining the health state of the patient based on the received values and a predefined state model that represents the health state of the patient by means of a state vector and using a plurality of model parameters, wherein the state vector comprises a glucose model value with information on the patient's glucose level; and
determining a deviation between the glucose measurement value and the glucose model value; and individually updating a model parameter for each patient based on the determined deviation.

15. The method according to claim 14, further comprising:
utilizing a computer program product comprising program code for performing the steps of the method when the program code is executed on a computer.
